(19) United States
(12) Patent Application Publication
(10) Pub. No.: US 2008/0305171 A1
(43) Pub. Date: Dec. 11, 2008

(54) PYRROLOPYRAZINE, FORMULATIONS, METHODS OF MANUFACTURE, AND METHODS OF USE THERE

(76) Inventors: Kristin Anne Arnold, Morrisville, PA (US); Jie Du, Lansdale, PA (US); Kurt Nielsen, Chadds Ford, PA (US); Hongmei M. Sheridan, Jackson, NJ (US); Shawn M. Watson, Cherry Hill, NJ (US)

Correspondence Address: CANTOR COLBURN, LLP, 20 Church Street, 22nd Floor, Hartford, CT, 06103, US

(21) Appl. No.: 12/128,776

(22) Filed: May 29, 2008

Related U.S. Application Data
(60) Provisional application No. 60/942,503, filed on Jun. 7, 2007.

(51) Int. Cl. A61K 31/4985; C07D 487/04; A61K 9/14; A61P 25/20

(52) U.S. Cl. 424/489; 544/350; 514/250

(57) ABSTRACT

Disclosed herein is a pyrrolopyrazine COMPOUND I having defined amounts of R isomer, particle size, and stability. Also disclosed are pyrrolopyrazine oral dosage forms comprising the described COMPOUND I material as well as methods of treating disorders amenable to therapy using COMPOUND I.

### CROSS REFERENCE TO RELATED APPLICATION

This application claims the benefit of U.S. Provisional Patent Application Ser. No. 60/942,503 filed Jun. 7, 2007, which is fully incorporated herein by reference.

### FIELD OF THE INVENTION

This invention pertains to pyrrolopyrazine COMPOUND I

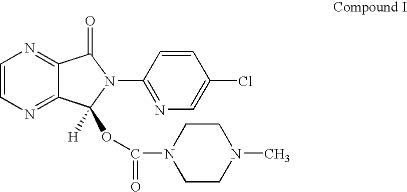

and pharmaceutical formulations comprising COMPOUND I or a pharmaceutically acceptable salt thereof, methods of manufacture and methods of use thereof.

### BACKGROUND

Pyrrolopyrazine COMPOUND II,

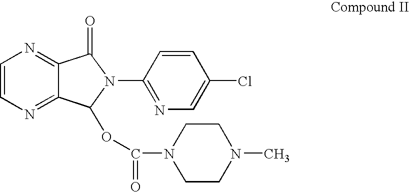

6-(5-chloro-2-pyridyl)-5-[(4-methyl-1-piperazinyl)carbonyloxy]-7-oxo-6,7-dihydro-5H-pyrrolo[3,4-b]pyrazine and its salts are pharmaceutical agents used in the treatment of sleep disorders, such as insomnia, and convulsive disorders, such as epilepsy.

COMPOUND II has an asymmetric carbon atom at the 5-position of the 5H-pyrrolo(3,4-b)-pyrazine ring-system, and as a result, exhibits optical isomerism.

Although racemic COMPOUND II, that is a mixture containing equal amounts of the S and R isomers, has been used to treat the above-described disorders, it has a low therapeutic index and also causes adverse effects. These adverse effects include, but are not limited to, the development of a bitter taste due to the salivary secretion of the active agent, dry mouth, drowsiness, morning tiredness, headache, dizziness, impairment of psychomotor skills and related effects.

In animals, the COMPOUND I displays hypnotic, sedative, anxiolytic, muscle-relaxant and anticonvulsant properties. Additionally, COMPOUND I is pharmaceutically more potent and has less adverse effects compared to the R isomer.

Oral dosage forms containing COMPOUND I are available to treat disorders such as insomnia. However, environmental conditions, such as elevated temperature and pH in an aqueous environment, can degrade and racemize COMPOUND I. See, Chirality, Volume 7, Issue 4, pages 267-271 (1995). Thus, it would be desirable to have COMPOUND I compositions that minimize conversion of the S isomer to the R isomer and to minimize any degradation products.

Additionally, as COMPOUND I is very slightly soluble in water, certain oral dosage forms of COMPOUND I may exhibit slow and incomplete dissolution and subsequent absorption of the active agent by the patient, thus leading to in vivo pharmacokinetic variability (bio-variability).

There remains a need in the art for COMPOUND I and its pharmaceutical formulations that have a wide range of release profiles, a more reliable dose-to-dose release, reduced pharmacokinetic variability, and isomeric and chemical stability over prolonged periods of time.

### SUMMARY

In one embodiment, COMPOUND I,

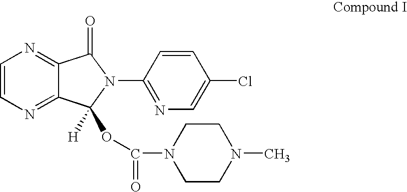

or a pharmaceutically acceptable salt thereof, comprises 0.3 to about 1% of the R isomer based on the total amount of S and R isomers or pharmaceutically acceptable salt thereof.

In another embodiment, COMPOUND I,

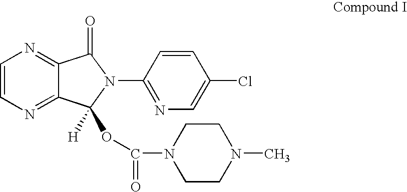

or a pharmaceutically acceptable salt thereof, comprises 0.3 to about 1% of the R isomer based on the total amount of S and R isomers or pharmaceutically acceptable salt thereof, wherein the amount of R isomer present in the COMPOUND I remains substantially unchanged between an initial time point and after storage of the COMPOUND I at about 25° C. and about 60% relative humidity for 12 months; an initial time point and after storage of the COMPOUND I at about 40° C. and about 75% relative humidity for 30 days; an initial time point and after storage of the COMPOUND I at about 40° C. and about 75% relative humidity for 60 days; an initial time point and after storage of the COMPOUND I at about 40° C. and about 75% relative humidity for 90 days; or an initial time point and after storage of the COMPOUND I at about 40° C. and about 75% relative humidity for 180 days or greater.

In another embodiment, COMPOUND I,

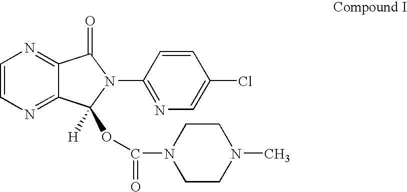

or a pharmaceutically acceptable salt thereof, comprises 0.3 to about 1% of the R isomer based on the total amount of S and R isomers or pharmaceutically acceptable salt thereof, wherein the COMPOUND I has an average particle size about 0.1 to about 500 micrometers.

In yet another embodiment, an oral dosage form, comprises a therapeutically effective amount of COMPOUND I, or a pharmaceutically acceptable salt thereof, comprising 0.3 to about 1% of the R isomer based on the total amount of S and R isomers or pharmaceutically acceptable salt thereof; and a pharmaceutically acceptable excipient.

In another embodiment, an oral dosage form, comprises a therapeutically effective amount of COMPOUND I, or a pharmaceutically acceptable salt thereof, comprising 0.3 to about 1% of the R isomer based on the total amount of S and R isomers or pharmaceutically acceptable salt thereof; and a pharmaceutically acceptable excipient; wherein the amount of R isomer present in the COMPOUND I remains substantially unchanged between an initial time point and after storage of the COMPOUND I at about 25° C. and about 60% relative humidity for 12 months; an initial time point and after storage of the COMPOUND I at about 40° C. and about 75% relative humidity for 30 days; an initial time point and after storage of the COMPOUND I at about 40° C. and about 75% relative humidity for 60 days; an initial time point and after storage of the COMPOUND I at about 40° C. and about 75% relative humidity for 90 days; or an initial time point and after storage of the COMPOUND I at about 40° C. and about 75% relative humidity for 180 days or greater.

In still yet another embodiment, an oral dosage form, comprises a therapeutically effective amount of COMPOUND I, or a pharmaceutically acceptable salt thereof, comprising 0.3 to about 1% of the R isomer based on the total amount of S and R isomers or pharmaceutically acceptable salt thereof; and a pharmaceutically acceptable excipient; wherein the COMPOUND I has an average particle size about 0.1 to about 500 micrometers.

In one embodiment, an oral dosage form, comprises a therapeutically effective amount of COMPOUND I, or a pharmaceutically acceptable salt thereof, comprising 0.3 to about 1% of the R isomer based on the total amount of S and R isomers or pharmaceutically acceptable salt thereof; and a pharmaceutically acceptable excipient; wherein the amount of R isomer present in the COMPOUND I remains substantially unchanged between an initial time point and after storage of the COMPOUND I at about 25° C. and about 60% relative humidity for 12 months, and wherein the COMPOUND I has an average particle size about 0.1 to about 500 micrometers.

These and other embodiments, advantages and features of the present invention become clear when detailed description and examples are provided in subsequent sections.

### DETAILED DESCRIPTION

Disclosed herein is a pyrrolopyrazine material, specifically COMPOUND I, or a pharmaceutically acceptable salt thereof, comprising 0.3 to about 1% of the R isomer based on the total amount of S and R isomers or pharmaceutically acceptable salt thereof. The COMPOUND I also exhibits a stability such that the amount of R isomer present in the COMPOUND I remains substantially unchanged between an initial time point and after storage of the COMPOUND I in a particular environment of temperature and humidity. Furthermore, the COMPOUND I material disclosed herein is prepared into specified particle size distributions for use in pharmaceutical dosage formulations.

The terms “COMPOUND I,” “the S isomer of COMPOUND II,” and “COMPOUND II S isomer” are used herein interchangeably and means (S)-6-(5-chloro-2-pyridyl)-5-[(4-methyl-1-piperazinyl)carbonyloxy]-7-oxo-6,7-dihydro-5H-pyrrolo[3,4-b]pyrazine or (+)-(5S)-6-(chloropyridin-2-yl)-7-oxo-6,7-dihydro-5H-pyrrolo[3,4-b]pyrazin-5-yl 4-methylpiperazine-1-carboxylate, including any solvates, hydrates, crystalline forms, non-crystalline forms, co-crystals, and polymorphs thereof unless otherwise stated.

Disclosed herein is a material, specifically COMPOUND I, or a pharmaceutically acceptable salt thereof, comprising 0.25 to about 1% of the R isomer based on the total amount of S and R isomers or pharmaceutically acceptable salt thereof, specifically about 0.3 to about 0.9%, and more specifically about 0.4 to about 0.8%. The determination of the amount of S and R isomers present in the COMPOUND I material can be determined using equipment and processes well known in the art. Exemplary processes include high performance liquid chromatography (HPLC) using chiral columns, capillary electrophoresis (CE), and the like. Specific procedures for capillary electrophoresis can be found in USP <727>.

The COMPOUND I can also exhibit an assay value of about 95% to about 105% calculated as anhydrous freebase, specifically about 97% to 103%, more specifically 98% to 102%, when determined with a suitable analytical method, specifically a chromatographic technology with a suitable detection means, more specifically by a HPLC method or its alternative, i.e., thin layer chromatography (TLC), titration, and CE.

“Pharmaceutically acceptable salts” include derivatives of COMPOUND I, wherein the COMPOUND I is modified by making acid addition salts thereof, and further refers to pharmaceutically acceptable solvates, including hydrates, crystalline forms, non-crystalline forms, and polymorphs of such salts. Examples of pharmaceutically acceptable salts include, but are not limited to, mineral or organic acid addition salts of basic residues. The pharmaceutically acceptable salts include salts and the quaternary ammonium salts of COMPOUND I. For example, acid salts include those derived from inorganic acids such as hydrochloric, hydrobromic, sulfuric, sulfamic, phosphoric, nitric and the like and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, theophyllineacetate, HOOC—(CH2)n-COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparaginate, glutamate, and the like; and combinations comprising one or more of the foregoing salts.

COMPOUND I can be in the form of a co-crystal. “Co-crystal” means a multi-component crystalline material containing COMPOUND I and one or more other components which are solid at room temperature.

Also provided herein are oral dosage forms comprising COMPOUND I or a pharmaceutically acceptable salt thereof, comprising 0.25 to about 1% of the R isomer based on the total amount of S and R isomers or pharmaceutically acceptable salt thereof, specifically about 0.3 to about 0.9%, and more specifically about 0.4 to about 0.8%.

In one embodiment, the COMPOUND I remains stable when stored under ambient temperatures and humidity for extended periods of time. The amount of R isomer present in the COMPOUND I remains substantially unchanged between an initial time point and after storage of the COMPOUND I at about 25° C. and about 60% relative humidity for 12 months, specifically 24 months, or longer. In another embodiment, the COMPOUND I exhibits a stability such that the amount of R isomer present in the COMPOUND I remains substantially unchanged between an initial time point and after storage of the COMPOUND I at about 40° C. and about 75% relative humidity for 30 days, specifically 60 days, more specifically 90 days, and yet more specifically 180 days.

In another embodiment, the amount of R isomer varies by less than about 2%, between an initial time point and after storage of the COMPOUND I at about 25° C. and about 60% relative humidity for 12 months, specifically about 24 months. In yet another embodiment, the amount of R isomer varies by less than about 2%, between an initial time point and after storage of the COMPOUND I at about 40° C. and about 75% relative humidity for 30 days, specifically 60 days, more specifically 90 days, and yet more specifically 180 days.

In still another embodiment, the amount of R isomer varies by less than about 0.001% to about 1%, more specifically less than about 0.01% to about 0.5%, and yet more specifically less than about 0.05% to about 0.1%, between an initial time point and after storage of the COMPOUND I at about 25° C. and about 60% relative humidity for 12 months, specifically about 24 months. In yet another embodiment, the amount of R isomer varies by less than about 0.001% to about 1%, more specifically less than about 0.01% to about 0.5%, and yet more specifically less than about 0.05% to about 0.1%, between an initial time point and after storage of the COMPOUND I at about 40° C. and about 75% relative humidity for 30 days, specifically 60 days, more specifically 90 days, and yet more specifically 180 days.

Also provided herein are oral dosage forms comprising COMPOUND I or a pharmaceutically acceptable salt thereof wherein the COMPOUND I within the oral dosage form also exhibits a stability such that the amount of R isomer present in the COMPOUND I remains substantially unchanged between an initial time point and after storage of the COMPOUND I under the temperature and humidity conditions previously described.

It has been determined that COMPOUND II both degrades and racemizes in ethanol:phosphate buffer solution with increasing temperature and pH. Accordingly, COMPOUND I is prepared and formulated under conditions to minimize both heat history and exposure to high pH, specifically by maintaining a pH of about 6.0 to about 8.0, and more specifically about 6.5 to about 7.5 when processed and prepared into dosage forms.

In another embodiment, the COMPOUND I material, or a pharmaceutically acceptable salt thereof, comprises not more than (NMT) about 0.15 percent single unknown impurity based on the total weight of the compound, specifically NMT about 0.10 percent, and yet more specifically NMT about 0.09 percent. In yet another embodiment, the total amount of impurities and R isomer is about 0.3 to about 5 weight percent based on the total weight of the compound, specifically about 0.3 to about 4 percent, and yet more specifically about 0.3 to about 3 percent. Other known impurities, excluding the R isomer, can be present at not more than 4.75 percent based on the total weight of the compound, specifically NMT about 4.5 percent, and more specifically NMT about 4.0 percent.

Furthermore, to provide reliable dose-to-dose release profiles of the oral dosage forms, including dissolution profiles, and thus to reduce in vivo pharmacokinetic variability, the COMPOUND I can be prepared to meet a predetermined particle size distribution.

In one embodiment, the COMPOUND I has an average particle size of about 0.1 to about 500 micrometers, specifically about 1 to about 250 micrometers, and yet more specifically about 10 to about 150 micrometers. The particle size distribution can have a D90 (meaning 90% of the particles are) under 100 micrometers, a D50 (meaning 50% of the particles are) under 50 micrometers, and a D10 (meaning 10% of the particles are) under 10 micrometers. The particle size can be determined using equipment and techniques well known in the art including for example light scattering (“laser diffraction”) techniques, static or dynamic; sieving; microscopy such as Scanning electron microscopy (SEM) and Environmental scanning electron microscopy (ESEM). An exemplary method used to determine particles size by light diffraction includes U.S. Pharmacopeia (USP)<429>.

Methods of preparing COMPOUND I having narrow particle size distributions include those well known in the art including milling with milling media, formation of a solution of COMPOUND I followed by spray drying, and the like. Specific micronization mills include bead mills, spiral jet and opposed jet mills.

In one embodiment, the COMPOUND I can exist in various forms of particles such as, for example, crystals, granules, microgranules, powders, pellets, amorphous solids, amorphous dispersions, or precipitates.

Methods for preparing COMPOUND II can be found in, for example, U.S. Pat. No. 3,862,149. COMPOUND I can be prepared from racemic COMPOUND II according to methods known in the art, such as chiral-phase chromatography, resolution of an optically active salt, stereoselective enzymatic catalysis by means of an appropriate microorganism, or asymmetric synthesis. Methods for preparing COMPOUND I can also be found in, for example “Stereochemistry of Carbon Compounds,” by E. L. Eliel (McGraw Hill, 1962) and Lochmuller C. H. et al., J. Chromatogr., 113:(3) 283-302 (1975).

The COMPOUND I material can be formulated into dosage forms suitable for administration via oral, buccal, transdermal, or injectable routes, specifically solid oral dosage forms for oral administration. A “dosage form” means a unit of administration of an active agent. Examples of dosage forms include tablets, capsules, injections, suspensions, liquids, emulsions, creams, ointments, lotions, suppositories, inhalable forms, transdermal forms, implants, and the like. The tablets can include orally disintegrating forms, chewable forms, compressed forms, monolithic forms, layered forms, etc.

The dosage forms can be formulated to have a particular release profile.

By “releasable form” is meant to include immediate-release, controlled-release, and extended-release forms. Certain release forms can be characterized by their dissolution profile. Dissolution profile as used herein, means a plot of the amount of active ingredient released as a function of time. The dissolution profile may be measured utilizing the Drug Release Test <724>, which incorporates standard test USP 30 (Test <711>) or by other test methods or conditions. A profile is characterized by the test conditions selected. Thus the dissolution profile can be generated at a preselected apparatus type, shaft speed, temperature, volume, and pH of the dissolution media.

A first dissolution profile can be measured at a pH level approximating that of the stomach. A second dissolution profile can be measured at a pH level approximating that of one point in the intestine or several pH levels approximating multiple points in the intestine.

A highly acidic pH may simulate the stomach and a less acidic to basic pH may simulate the intestine. By the term “highly acidic pH”: it is meant a pH of about 1 to about 4. By the term “less acidic to basic pH” is meant a pH of greater than about 4 to about 7.5, specifically about 6 to about 7.5. A pH of about 1.2 can be used to simulate the pH of the stomach. A pH of about 6 to about 7.5, specifically about 6.8, can be used to simulate the pH of the intestine.

By “immediate-release”, it is meant a conventional or non-modified release in which greater than or equal to about 75% of the active agent is released within two hours of administration, specifically within one hour of administration. Alternatively, an “immediate-release” formulation contains substantially no added release retarding agents.

By “controlled-release” it is meant a dosage form in which the release of the active agent is controlled or modified over a period of time. Controlled can mean, for example, extended- or delayed-release at a particular time. Alternatively, controlled can mean that the release of the active agent is extended for longer than it would be in an immediate-release dosage form, i.e., at least over several hours.

“Sustained-release” or “extended-release” include the release of the active agent at such a rate that blood (e.g., plasma) levels are maintained within a therapeutic range for at least about 8 hours, specifically at least about 12 hours, and more specifically at least about 24 hours after administration at steady-state. The term steady-state means that a plasma level for a given active agent has been achieved and which is maintained with subsequent doses of the drug at a level which is at or above the minimum effective therapeutic level for a given active agent.

By “delayed-release”, it is meant that there is a time-delay before significant plasma levels of the active agent are achieved. A delayed-release formulation of the active agent can avoid an initial burst of the active agent, or can be formulated so that release of the active agent in the stomach is reduced and absorption occurs in the small intestine.

By “oral dosage form” is meant to include a unit dosage form for oral administration. Exemplary oral dosage forms include a tablet and a capsule. An oral dosage form may optionally comprise a plurality of subunits such as, for example, microcapsules or microtablets. Multiple subunits may be packaged for administration in a single dose. By “subunit” is meant to include a composition, mixture, particle, pellet, etc., that can provide an oral dosage form alone or when combined with other subunits.

The oral dosage form comprises a therapeutically effective amount of COMPOUND I. The magnitude of a prophylactic or therapeutic dose of COMPOUND I in the acute or chronic management of disease, such as, for example, sleep disorders (e.g., insomnia), convulsive disorders (e.g., epilepsy), can vary with the severity of the condition to be treated. The dose, and/or the dose frequency, can also vary according to the age, body weight, and response of the individual patient. In general, the total daily dose ranges is about 1.5 mg to about 15 mg, specifically about 2.5 mg to about 12.5 mg, and more specifically about 3.5 mg to about 10.0 mg. In managing a patient, the therapy can be initiated at a lower dose, for example, about 2.5 mg to about 7.5 mg and increased up to about 10 mg or higher depending on the patient's global response. Further, for children and patients over 65 years, and those with impaired renal or hepatic function, an initial low dose can be administered, and that they be titrated based on global response and blood level. It may be necessary to use dosages outside these ranges in some cases.

The oral dosage form includes, in addition to COMPOUND I, a pharmaceutically acceptable excipient. Excipients may be added to facilitate manufacture, enhance stability, control release, enhance product characteristics, enhance bioavailability, enhance patient acceptability, etc. Pharmaceutical excipients include, for example, a filler/diluent, a binder, a disintegrant, a lubricant, a glidant, a compression aid, a colorant, a sweetener, a preservative, a suspending agent, a dispersing agent, a film former, a flavor, printing ink, etc.

The oral dosage form can comprise a filler (“diluent”), such as a water insoluble filler, water soluble filler, and combinations thereof. The filler may be a water insoluble filler, such as calcium phosphate, silicon dioxide, titanium dioxide, talc, alumina, starch, kaolin, polacrilin potassium, powdered cellulose, microcrystalline cellulose, and combinations comprising one or more of the foregoing fillers. Exemplary water-soluble fillers include water soluble sugars and sugar alcohols, specifically lactose, glucose, fructose, sucrose, mannose, dextrose, galactose, the corresponding sugar alcohols and other sugar alcohols, such as mannitol, sorbitol, xylitol, and combinations comprising one or more of the foregoing fillers.

The amount of filler present in the oral dosage form can be about 1 wt % to about 98 wt % based on the total weight of the dosage form, specifically about 5 wt % to about 85 wt %, and more specifically about 25 wt % to about 65 wt %.

Binders hold the ingredients in the dosage form together. Exemplary binders include, for example, polyvinyl pyrrolidone, hydroxypropyl cellulose, hydroxypropyl methylcellulose, methylcellulose and hydroxyethyl cellulose, sugars, and combinations comprising one or more of the foregoing binders.

The amount of binder present in the oral dosage form can be about 1 wt % to about 98 wt % based on the total weight of the dosage form, specifically about 5 wt % to about 85 wt %, and more specifically about 25 wt % to about 65 wt %.

Disintegrants expand when wet causing a tablet to break apart. Exemplary disintegrants include water swellable substances, for example, low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose (sodium croscarmellose), e.g. Ac-di-sol®, Primellose®; sodium starch glycolate, e.g. Primojel®; sodium carboxymethylcellulose, e.g. Nymcel ZSB10®; sodium carboxymethyl starch, e.g. Explotab®; ion-exchange resins, e.g. Dowex® or Amberlite®; microcrystalline cellulose, e.g. Avicel®; starches and pregelatinized starch, e.g. Starch 1500®, Sepistab ST200®; formalin-casein, e.g. Plas-Vita®, and combinations comprising one or more of the foregoing water swellable substances.

The amount of disintegrant present in the oral dosage form can be about 1 wt % to about 30 wt % based on the total weight of the dosage form, specifically about 5 wt % to about 20 wt %, and more specifically about 10 wt % to about 15 wt %.

Lubricants, for example, aid in the processing of powder materials. Exemplary lubricants include calcium stearate, glycerol behenate, magnesium stearate, mineral oil, a polyethylene glycol, sodium stearyl fumarate, stearic acid, talc, vegetable oil, zinc stearate, and combinations comprising one or more of the foregoing lubricants.

The amount of lubricant present in the oral dosage form can be about 0.001 wt % to about 10 wt % based on the total weight of the dosage form, specifically about 0.01 wt % to about 8 wt %, and more specifically about 0.1 wt % to about 5 wt %.

Glidants include, for example, silicon dioxide, specifically colloidal silicon dioxide.

Preservatives help to preserve shelf life of the oral dosage form. For example, preservatives can help to the COMPOUND I to remain substantially stable when stored for long periods of time. Suitable preservatives include antioxidants such as, for example, potassium metabisulfite, ascorbic acid, butylhydroxytoluene, butylhydroxyanisole, tocopherol.

COMPOUND I is slightly soluble in water, which may limit its absorption in the gastrointestinal (“GI”) tract. In one embodiment, the oral dosage form optionally comprises a solubilizer to enhance the solubility of the COMPOUND I, thus its absorption in the GI tract. By “solubilizer” is meant to include additives to increase the solubility of the COMPOUND I in water.

Suitable solubilizers for use in the oral dosage forms include, but not limit to: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropylmethyl cellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, epsilon-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon-caprolactone and isomers thereof, delta-valerolactone and isomers thereof, gamma-butyrolactone and isomers thereof; and other solubilizers known in the art, such as dimethyl acetamide, dimethyl isosorbide (Arlasolve DMI (ICI)), N-methylpyrrolidones (Pharmasolve (ISP)), monooctanoin, diethylene glycol monoethyl ether (available from Gattefosse under the trade name Transcutol), and combinations comprising one or more of the forgoing solubilizers.

The oral dosage form can optionally comprise a coating to function as a protective layer, identification, aesthetics, etc. The coating can be a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

In one embodiment, the oral dosage form comprises a film coat. Suitable film forming polymers include, but not limit to, cellulose ether, such as methyl cellulose, ethylcellulose, hydroxypropyl cellulose, hydroxypropylmethyl cellulose, hydroxybutyl methyl cellulose; cellulose ester, such as cellulose acetate, cellulose propionate, cellulose acetate propionate, cellulose acetate butyrate, cellulose acetate phthalate, carboxymethyl cellulose, cellulose triacetate, cellulose sulphate sodium salt; poly(methyl methacrylate), poly(ethyl methacrylate), poly(butyl methacrylate), poly(isobutyl methacrylate), poly(hexyl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene)high density, (poly propylene), poly(ethylene glycol poly(ethylene oxide), poly(ethylene terephthalate), poly(vinyl alcohol), poly(vinyl isobutyl ether), poly(viny acetate), poly(vinyl chloride), polyvinyl pyrrolidone, and combinations comprising one or more of the foregoing polymers.

The film coat optionally comprises a plasticizer. Suitable plasticizers include, for example, for ethyl cellulose and other celluloses plasticizers such as dibutyl sebacate, diethyl phthalate, triethyl citrate, tributyl citrate, triacetin, and combinations comprising one or more of the foregoing plasticizers, although it is possible that other water-insoluble plasticizers (such as acetylated monoglycerides, phthalate esters, castor oil, etc.) can be used. Suitable plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, and combinations comprising one or more of the foregoing plasticizers, although it is possible that other plasticizers (such as acetylated monoglycerides, phthalate esters, castor oil, etc.) can be used.

The oral dosage form can optionally comprise compression aids, colors, sweeteners, preservatives, suspending agents, dispersing agents, flavors, printing inks, etc.

In one embodiment, the COMPOUND I comprised in the oral dosage form in the form of its neutral or salt form and can be in various forms of particles, such as, for example, crystals, co-crystals, granules, microgranules, powders, pellets, amorphous solids, amorphous dispersions, or precipitates. The COMPOUND I particles can have a defined particle size distribution as defined above. The defined particle size distribution can provide benefits, such as a more reliable dose-to-dose release and reduced bio-variability between doses.

COMPOUND I can be formulated into an orally disintegrating tablet that is a non-chewable, fast dissolving dosage form. In one embodiment, “orally disintegrating tablet” means a solid dosage form which disintegrates rapidly when placed upon the tongue to leave an easily swallowable residue. The tablet can disintegrate within 2 minutes, specifically within 1 minute, and yet more specifically within 30 seconds from being place on the tongue. Orally disintegrating tablet technology is known to those of ordinary skill in the art and can be used herein. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. If microparticles or other discrete subunits are present in the dosage form, disintegration of these are not to be included in the disintegration time. In another embodiment, disintegration can be measured according to USP <701>

Exemplary orally disintegrating tablet formulations include Zydis by Eli Lilly. Zydis is a rapidly dissolvable, freeze-dried, sugar matrix formulated as a rapidly dissolving tablet. U.S. Pat. No. 5,178,878 and U.S. Pat. No. 6,221,392 provide teachings regarding fast-dissolve dosage forms.

Other orally disintegrating tablet technologies include DuraSolv® by CIMA Labs, Eden Prairie, Minn.; OraSolv® by CIMA Labs; WOWTAB® by Yamanouchi, Norman, Okla., Pharmaburst™ by SPI Pharma, NanoCrystal® Nanomelt™ by Elan, FlashDose by Fuisz Technologies, Ltd., Flashtab by Prographarm Group, and OraQuick by KV Pharmaceutical Co., Inc.

In one embodiment, the orally disintegrating tablet includes a water or saliva activated effervescent disintegrant and particles containing COMPOUND I. The particles contain COMPOUND I optionally together with a protective material substantially encompassing the active agent to substantially shield COMPOUND I from contact with the environment outside of the particle. For example, each particle can contain COMPOUND I and optionally other pharmaceutically acceptable excipients, wherein the particle is coated with a protective coating. In another example, particles containing COMPOUND I are dispersed or dissolved in a matrix of a protective material rather than coated with a protective material.

The particles are then mixed with the water or saliva activated effervescent disintegrant and formed into a tablet. The water or saliva activated effervescent disintegrant is present in an amount effective to aid in disintegration of the tablet, and to provide a distinct sensation of effervescence when the tablet is placed in the mouth of a patient. Upon disintegration of the tablet, the particles are released and can be swallowed as a slurry or suspension.

The water or saliva activated effervescent disintegrant includes compounds which evolve gas, specifically by means of chemical reactions which take place upon exposure of the water or saliva activated effervescent disintegrant to water and/or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Exemplary acid sources or acid are those which are safe for human consumption such as food acids, acid anhydrides and acid salts. Food acids include citric acid, tartaric acid, malic acid, fumaric acid, adipic acid, and succinic acid, and combinations comprising one or more of the foregoing carbonates. Acid anhydrides of the above-described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, and combinations comprising one or more of the foregoing carbonates.

The amount of water or saliva activated effervescent disintegrant useful for the formation of the orally disintegrating tablets is about 5 to about 50 weight percent based on the total weight of the tablet, specifically about 15 and about 30 weight percent, and more specifically about 20 to about 25 weight percent.

The protective material substantially encompassing the active agent to substantially shield COMPOUND I from contact with the environment outside of the particle can include a coating material such as alkylacrylate copolymers, shellac, zein, other coatings described herein, and the like.

In one embodiment, disintegration time in the mouth can be measured according to USP <701> by observing the disintegration time of the tablet in water or another medium at about 37±2° C. The disintegration medium can be purified water, 0.1N HCl, 0.1N NaOH, 7.5 pH buffer, 6.8 pH buffer, 0.5% sodium dodecyl sulfate, and the like.

In one embodiment, a dosage form containing COMPOUND I disintegrates in less than 5 minutes in purified water or 0.1N HCl when tested according to USP <701>, specifically less than about 4 minutes, and yet more specifically less than about 3 minutes.

In another embodiment, the orally disintegrating dosage form containing COMPOUND I disintegrates in less than 1 minute in purified water or 0.1N HCl when tested according to USP <701>, specifically less than about 45 seconds, and yet more specifically less than about 30 seconds.

COMPOUND I can also be formulated into controlled-release dosage forms. Exemplary forms include polymeric matrices containing COMPOUND I, coated tablets, coated particles, and the like. Generally, an extended-release dosage form comprises a release-retarding material. The release-retarding material can be, for example, in the form of a matrix or a coating. The COMPOUND I in extended-release form may be, for example, a particle of COMPOUND I that is combined with a release-retarding material. The release-retarding material is a material that permits release of the active agent at a sustained rate in an aqueous medium. The release-retarding material can be selectively chosen so as to achieve, in combination with the other stated properties, a desired in vitro release rate.

Release-retarding materials include, for example acrylic polymers, alkylcelluloses, shellac, zein, hydrogenated vegetable oil, hydrogenated castor oil, polyvinylpyrrolidine, vinyl acetate copolymers, polyethylene oxide, and a combination comprising at least one of the foregoing materials. The extended-release oral dosage form can contain between about 1 wt % and about 80 wt % of the release-retarding material based on the total weight of the oral dosage form.

Suitable acrylic polymers that can be used as release-retarding materials include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and a combination comprising at least one of the foregoing polymers. The acrylic polymer may comprise methacrylate copolymers described in NF XXIV as fully polymerized copolymers of acrylic and methacrylic acid esters with a low content of quaternary ammonium groups.

Suitable alkylcelluloses include, for example, methyl cellulose, ethylcellulose, and the like. Those skilled in the art will appreciate that other cellulosic polymers, including other alkyl cellulosic polymers, can be substituted for part or all of the ethylcellulose.

Other suitable release-retarding materials include neutral or synthetic waxes, fatty alcohols (such as lauryl, myristyl, stearyl, cetyl or specifically cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and a combination comprising at least one of the foregoing materials. Suitable waxes include beeswax, glycowax, castor wax, carnauba wax and wax-like substances, e.g., material normally solid at room temperature and having a melting point of from about 30° C. to about 100° C., and a combination comprising at least one of the foregoing waxes.

In other embodiments, the release-retarding material may comprise digestible, long chain (e.g., C₈-C₅₀, specifically C₁₂-C₄₀), substituted or unsubstituted hydrocarbons, such as fatty acids, fatty alcohols, glyceryl esters of fatty acids, mineral and vegetable oils, waxes, and a combination comprising at least one of the foregoing materials. Hydrocarbons having a melting point of between about 25° C. and about 90° C. may be used. Specifically, long chain hydrocarbon materials, fatty (aliphatic) alcohols can be used. The oral dosage form can contain up to about 60 wt % of a digestible, long chain hydrocarbon, based on the total weight of the oral dosage form.

Further, the extended-release matrix can contain up to about 60 wt % of a polyalkylene glycol.

Alternatively, the release-retarding material may comprise polylactic acid, polyglycolic acid, or a co-polymer of lactic and glycolic acid.

Alternatively, the release-retarding material can include, for example, crosslinked sodium carboxymethylcellulose, crosslinked hydroxypropylcellulose, high molecular weight hydroxypropylmethylcellulose, carboxymethyl starch, potassium methacrylate/divinylbenzene copolymer, polymethylmethacrylate, crosslinked polyvinylpyrrolidone, high molecular weight polyvinylalcohols, methylcellulose, carboxymethylcellulose, low molecular weight hydroxypropylmethylcellulose, low molecular weight polyvinylalcohols, polyethylene glycols, non-crosslinked polyvinylpyrrolidone, medium viscosity hydroxypropylmethylcellulose, medium viscosity polyvinylalcohols, combinations thereof and the like.

Release-modifying agents, which affect the release properties of the release-retarding material, can optionally be used. The release-modifying agent can, for example, function as a pore-former. The pore former can be organic or inorganic, and include materials that can be dissolved, extracted or leached from the material in the environment of use. The pore-former can comprise one or more hydrophilic polymers, such as hydroxypropylmethylcellulose, hydroxypropylcellulose, polycarbonates comprised of linear polyesters of carbonic acid in which carbonate groups reoccur in the polymer chain, and a combination comprising at least one of the foregoing release-modifying agents. Alternatively, the pore-former may be a small molecule such as lactose, or metal stearates, and a combination comprising at least one of the foregoing release-modifying agents.

The release-retarding material can also optionally include other additives such as an erosion-promoting agent (e.g., starch and gums); and/or a semi-permeable polymer. In addition to the above ingredients, an extended-release dosage form may also contain suitable quantities of other materials, e.g., diluents, lubricants, binders, granulating aids, colorants, flavorants and glidants that are conventional in the pharmaceutical art. The release-retarding material can also include an exit means comprising a passageway, orifice, or the like. The passageway can have any shape, such as round, triangular, square, elliptical, irregular, etc.

The extended-release dosage form comprising COMPOUND I or a salt thereof and a release-retarding material may be prepared by a suitable technique for preparing active agents as described in detail below. The COMPOUND I or a salt thereof and release-retarding material may, for example, be prepared by wet granulation techniques, melt extrusion techniques, etc. To obtain an extended-release dosage form, it may be advantageous to incorporate an additional hydrophobic material.

The COMPOUND I or salt thereof in extended-release form can include a plurality of substrates (particles such as microparticles) comprising the active agent, which substrates are coated with an extended-release coating comprising a release-retarding material. The extended-release preparations may thus be made in conjunction with a multiparticulate system, such as beads, ion-exchange resin beads, spheroids, microspheres, seeds, pellets, granules, and other multiparticulate systems in order to obtain a desired extended-release of the COMPOUND I or salt thereof. The multiparticulate system can be presented in a capsule or other suitable unit dosage form.

In certain cases, more than one multiparticulate system can be used, each exhibiting different characteristics, such as pH dependence of release, time for release in various media (e.g., acid, base, simulated intestinal fluid), release in vivo, size, and composition.

In some cases, a spheronizing agent, together with the COMPOUND I or salt thereof can be spheronized to form spheroids. Microcrystalline cellulose and hydrous lactose impalpable are examples of such agents. Additionally (or alternatively), the spheroids can contain a water insoluble polymer, specifically an acrylic polymer, an acrylic copolymer, such as a methacrylic acid-ethyl acrylate copolymer, or ethyl cellulose. In this formulation, the extended-release coating will generally include a water insoluble material such as a wax, either alone or in admixture with a fatty alcohol, or shellac or zein.

Spheroids or beads, coated with COMPOUND I or a salt thereof can be prepared, for example, by dissolving or dispersing the active agent in a solvent and then spraying the solution onto a substrate, for example, sugar spheres NF, 18/20 mesh, using a Wurster insert. Optionally, additional ingredients are also added prior to coating the beads in order to assist the COMPOUND I or salt thereof binding to the substrates, and/or to color the resulting beads, etc. The resulting substrate-active agent may optionally be overcoated with a barrier material, to separate the therapeutically active agent from the next coat of material, e.g., release-retarding material. For example, the barrier material is a material comprising hydroxypropylmethylcellulose. However, film-formers known in the art may be used.

To obtain a extended-release of COMPOUND I or salt thereof in a manner sufficient to provide a therapeutic effect for the sustained durations, the substrate comprising the active agent can be coated with an amount of release-retarding material sufficient to obtain a weight gain level from about 2 wt % to about 30 wt %, specifically about 5 wt % to about 25 wt %, and more specifically about 7 wt % to about 20 wt %, although the coat can be greater or lesser depending upon the physical properties of the active agent utilized and the desired release rate, among other things. Moreover, there can be more than one release-retarding material used in the coat, as well as various other pharmaceutical excipients.

The release-retarding material may thus be in the form of a film coating comprising a dispersion of a hydrophobic polymer. Solvents used for application of the release-retarding coating include pharmaceutically acceptable solvents, such as water, methanol, ethanol, methylene chloride, and a combination comprising at least one of the foregoing solvents.

In addition, the extended-release profile of COMPOUND I or salt thereof (either in vivo or in vitro) can be altered, for example, by using more than one release-retarding material, varying the thickness of the release-retarding material, changing the particular release-retarding material used, altering the relative amounts of release-retarding material, altering the manner in which the plasticizer is added (e.g., when the extended-release coating is derived from an aqueous dispersion of hydrophobic polymer), by varying the amount of plasticizer relative to retardant material, by the inclusion of additional ingredients or excipients, by altering the method of manufacture, etc.

The extended-release formulations slowly release COMPOUND I or salt thereof, e.g., when ingested and exposed to gastric fluids, and then to intestinal fluids. The extended-release profile of the formulations can be altered, for example, by varying the amount of retardant, e.g., hydrophobic material, by varying the amount of plasticizer relative to hydrophobic material, by the inclusion of additional ingredients or excipients, by altering the method of manufacture, etc.

Exemplary forms containing a release-retarding material coating can comprise COMPOUND I blended with a water soluble polymer that is a film forming polymer. Useful water soluble film forming polymers are polymers that have an apparent viscosity of 1 to 100 mPa·s when dissolved in a 2% aqueous solution at 20° C. solution. For example, the water soluble film forming polymers can be selected from the group comprising alkylcelluloses such as methylcellulose, hydroxyalkylcelluloses such as hydroxymethylcellulose, hydroxyethylcellulose, hydroxypropylcellulose and hydroxybutylcellulose, hydroxyalkyl alkylcelluloses such as hydroxyethyl methylcellulose and hydroxypropyl methylcellulose, carboxyalkylcelluloses such as carboxymethylcellulose, alkali metal salts of carboxyalkylcelluloses such as sodium carboxymethylcellulose, carboxyalkyl alkylcelluloses such as carboxymethyl ethylcellulose, carboxyalkylcellulose esters, starches, pectines such as sodium carboxymethylamylopectine, chitine derivates such as chitosan, polysaccharides such as alginic acid, alkali metal and ammonium salts thereof, carrageenans, galactomannans, traganth, agar-agar, gum arabicum, guar gum and xanthan gum, polyacrylic acids and the salts thereof, polymethacrylic acids and the salts thereof, methacrylate copolymers, polyvinylalcohol, polyvinylpyrrolidone, copolymers of polyvinylpyrrolidone with vinyl acetate, polyalkylene oxides such as polyethylene oxide and polypropylene oxide and copolymers of ethylene oxide and propylene oxide. Other pharmaceutically acceptable polymers that exhibit similar as defined above physico-chemical properties as defined above are equally suitable.

Specific water soluble film forming polymers are for example hydroxypropyl methylcellulose, polymethacrylate, hydroxypropylcellulose, or a polyvidone; more specifically hydroxypropyl methylcelluloses (HPMCs). HPMCs contain sufficient hydroxypropyl and methoxy groups to render it water-soluble. HPMC having a methoxy degree of substitution from about 0.8 to about 2.5 and a hydroxypropyl molar substitution from about 0.05 to about 3.0 are generally water-soluble. Methoxy degree of substitution refers to the average number of methyl ether groups present per anhydroglucose unit of the cellulose molecule. Hydroxypropyl molar substitution refers to the average number of moles of propylene oxide which have reacted with each anhydroglucose unit of the cellulose molecule. Suitable HPMC include those having a viscosity from about 1 to about 100 mPa·s, specifically about 3 to about 15 mPa·s, and more specifically about 5 mPa·s.

The weight-by-weight ratio of active agent:water soluble film forming polymer is in the range of about 17:1 to about 1:5, specifically about 10:1 to about 1:3, and more specifically about 7:1 to about 1:2.

The particles generally comprise (a) a central, rounded or spherical core, (b) a layer or a coating film of a water soluble film forming polymer and COMPOUND I or a salt thereof, (c) optionally a barrier polymer layer and (d) a release retarding material coating. The core can have a diameter of about 250 to about 2000 micrometers, specifically about 600 to about 1500 micrometers, and yet more specifically about 750 to about 1000 micrometers.

Materials suitable for use as the cores of the particles include pharmaceutically acceptable materials that have appropriate dimensions and firmness. Examples of such materials are polymers e.g. plastic resins; inorganic substances, e.g. silica, glass, hydroxyapatite, salts (sodium or potassium chloride, calcium or magnesium carbonate) and the like; organic substances, e.g. activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like saccharides.

The combination of the water soluble film forming polymer and COMPOUND I can be coated on the core as a layer to form a coated core.

In another embodiment, the cores themselves can contain COMPOUND I. The cores containing COMPOUND I can be granules or spheroids (spherical granules) prepared according to art-known methods of granulation and spheronization.

The particles can be filled in hard-gelatin capsules or blended with a compressible excipient and compressed into tablets such that a therapeutically effective amount of the active ingredient is available per dosage form. An desired pharmacokinetic profile (fast onset, level peak and trough values) can be obtained when about 60 to about 90 weight % of the COMPOUND I based on the total amount of COMPOUND I in the dosage form, specifically about 70 to about 80 weight % of the COMPOUND I is comprised within the controlled-release particles and about 10 to about 40 weight %, specifically about 20 to about 30 weight % of the COMPOUND I based on the total amount of COMPOUND I in the dosage form, is in an immediate-release form.

In order to achieve the desired pharmacokinetic profile, the dosage forms may be filled with particles that release COMPOUND I at different rates, a kind that releases COMPOUND I slowly, and a kind that releases COMPOUND I more rapidly, in particular one kind that releases the active ingredient immediately, e.g. particles as described that lack the release retarding material coating.

The different particles may be filled consecutively in the capsules, or they may be premixed and the thus obtained premix may be filled into the capsules (taking into account possible segregation).

Alternatively, the controlled-release particles may further comprise a top-coat of a water-soluble polymer as described hereinbefore and COMPOUND I which is released practically immediately upon ingestion and thus ensures a rapid onset of action.

In another embodiment, a capsule is filled with controlled-release particles as described above (about 60 to about 90 weight %, specifically about 70 to about 80 weight % based on the total weight of COMPOUND I in the dosage form) together with one or more minitablets which comprise the remaining amount of COMPOUND I.

The COMPOUND I formulations can be coated with a material to delay release of the COMPOUND I until the formulation is exposed to the intestinal tract. These formulations include enteric coated formulations, which are forms coated with a composition that is non-toxic and includes a pharmaceutically acceptable enteric polymer which is predominantly soluble in the intestinal fluid, but substantially insoluble in the gastric juices. An enteric coating is a coating that prevents release of the active agent until the dosage form reaches the small intestine. Enteric coated dosage forms comprise COMPOUND I or a salt thereof coated with an enteric polymer. Examples include polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), which is an anionic copolymer based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and the like, and a combination comprising at least one of the foregoing enteric polymers. Other examples include natural resins, such as shellac, SANDARAC, copal collophorium, and a combination comprising at least one of the foregoing polymers. Yet other examples of enteric polymers include synthetic resin bearing carboxyl groups. The methacrylic acid:acrylic acid ethyl ester 1:1 copolymer solid substance of the acrylic dispersion sold under the trade designation “EUDRAGIT L-100-55” may be suitable.

The extended-release COMPOUND I formulations can be prepared to include an immediate-release portion. An exemplary form may provide at least a part of the dose with an extended-release of COMPOUND I and another part of the formulation with rapid or immediate-release. The immediate- and extended-release of COMPOUND I can be achieved according to different principles, such as by single dose layered pellets or tablets, by multiple dose layered pellets or tablets, or by two or more different fractions of single or multiple dose layered pellets or tablets, optionally in combination with pellets or tablets having instant release. Multiple dose layered pellets may be filled into a capsule or together with tablet excipients compressed into a multiple unit tablet. Alternatively, a multiple dose layered tablet may be prepared.

Pellets or tablets may comprise a core material, optionally layered on a seed/sphere, the core material comprising COMPOUND I together with a water swellable substance; an optional intermediate layer surrounding the core; and an outer coating layer containing COMPOUND I in an immediate-release form. Alternatively, the layered pellets or tablets may comprise a core material comprising COMPOUND I; a surrounding layer comprising a water swellable substance; an outer coating layer containing COMPOUND I in an immediate-release form; and optional intermediate layers for ease of processing or improved dosage form stability.

In another embodiment, part of the COMPOUND I controlled-release dosage form is present in an immediate-release portion, for example, as particles lacking a release-retarding material coating, or as immediate-release minitablets, or as a topcoat on the controlled-release formulation.

Also disclosed herein are COMPOUND I oral dosage forms that are bioequivalent to the reference listed drug according to New Drug Application No. 021476. In one embodiment, the oral dosage form comprises COMPOUND I, or a pharmaceutically acceptable salt thereof, comprising 0.3 to about 1% of the R isomer based on the total amount of S and R isomers or pharmaceutically acceptable salt thereof; and is bioequivalent to a reference drug according to New Drug Application NO. 021476. “Reference drug” means an COMPOUND I product as described in U.S. Federal Food and Drug Administration's New Drug Application No. 021476 approved on Dec. 15, 2004 as provided in the U.S. Federal Food and Drug Administration's Orange Book, Approved Drug Products with Therapeutic Equivalence Evaluations. The formulations associated with New Drug Application NO. 021476 are oral tablets containing COMPOUND I at strengths of 1, 2, or 3 mg, which is marketed by Sepracor Inc. The 3 mg strength is the “reference listed drug” under 21 CFR 314.94(a)(3)), i.e., the listed drug identified by FDA as the drug product upon which an applicant relies in seeking approval of its ANDA.

The formulations described herein exhibit bioequivalence to the marketed drug product, for example the reference drug according to New Drug Application NO. 021476 and provide similar mean plasma concentrations when dosed under fed and fasting conditions. “Bioequivalence” means the absence of a significant difference in the rate and extent to which the active agent or surrogate marker for the active agent in pharmaceutical equivalents or pharmaceutical alternatives becomes available at the site of action when administered in an appropriately designed study.

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS-GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001, both of which are incorporated herein in their entirety.

In another embodiment, bioequivalence is determined according to the European Medicines Agency (EMEA) document “Note for Guidance on the Investigation of Bioavailability and Bioequivalence”, issued Jul. 26, 2001, available from EMEA.

In an embodiment, bioequivalence of a COMPOUND I oral dosage form to a reference drug is determined by an in vivo pharmacokinetic study to determine a pharmacokinetic parameter for the COMPOUND I oral dosage form. Specifically, bioequivalence can be determined by an in vivo pharmacokinetic study comparing a pharmacokinetic parameter for the two compositions. A pharmacokinetic parameter for the COMPOUND I oral dosage form or the reference drug can be measured in a single or multiple dose bioequivalence study using a replicate or a nonreplicate design. For example, the pharmacokinetic parameters for COMPOUND I oral dosage form of the present invention and for a reference drug can be measured in a single dose pharmacokinetic study using a two-period, two-sequence crossover design. Alternately, a four-period, replicate design crossover/or non-crossover study may also be used. Single doses of the test composition and reference drug are administered and blood or plasma levels of the active agent are measured over time. Pharmacokinetic parameters characterizing rate and extent of active agent absorption are evaluated statistically.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

Under U.S. FDA guidelines, two products (e.g. COMPOUND I oral dosage form and the reference drug according to New Drug Application NO. 021476) or methods (e.g., dosing under non-fasted versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

To show bioequivalence between two compositions or administration conditions pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In an embodiment, a given experiment, a COMPOUND I oral dosage form is considered to be bioequivalent to reference drug according to New Drug Application NO. 021476 if both the Test/Reference ratio for the geometric mean of logarithmic transformed AUC_{0-∞}, AUC₀₋ₜ, or Cₘₐₓ ratio along with its corresponding lower and upper 90% CI limits are within a lower limit of about 0.80 and an upper limit of about 1.25. Thus, for direct comparison between a COMPOUND I oral dosage form and reference drug according to New Drug Application NO. 021476, it is sometimes preferred to determine the pharmacokinetic parameters for the COMPOUND I oral dosage form and reference drug according to New Drug Application NO. 021476 side-by side in the same pharmacokinetic study.

In some embodiments a single dose pharmacokinetic study is performed under non-fasted or fasted conditions.

In other embodiments, the single dose pharmacokinetic study is conducted between the COMPOUND I oral dosage form and the reference listed drug using the strength specified by the FDA in APPROVED DRUG PRODUCTS WITH THERAPEUTIC EQUIVALENCE EVALUATIONS(ORANGE BOOK).

In some embodiments, an in vivo pharmacokinetic study is performed to compare all COMPOUND I oral dosage forms with corresponding strengths of drug according to New Drug Application NO. 021476 (e.g., 1, 2, or 3 mg referenced drug product). In other embodiments, an in vivo pharmacokinetic study is performed only for the COMPOUND I oral dosage form of the present invention at the strength of the reference listed drug product for reference drug according to New Drug Application NO. 021476 (the highest approved strength, or 3 mg as of Dec. 15, 2004) and at the other lower strengths, the COMPOUND I oral dosage forms meet the COMPOUND I dissolution test described herein.

In one embodiment, the oral dosage form comprises COMPOUND I, or a pharmaceutically acceptable salt thereof, comprising 0.3 to about 1% of the R isomer based on the total amount of S and R isomers or pharmaceutically acceptable salt thereof, wherein the oral dosage form exhibits a ratio of a geometric mean of logarithmic transformed AUC_{0-∞} of the oral dosage form to a geometric mean of logarithmic transformed AUC_{0-∞} of COMPOUND I reference drug of about 0.80 to about 1.25.

In another embodiment, the oral dosage form comprises COMPOUND I, or a pharmaceutically acceptable salt thereof, comprising 0.3 to about 1% of the R isomer based on the total amount of S and R isomers or pharmaceutically acceptable salt thereof, wherein the oral dosage form exhibits a ratio of a geometric mean of logarithmic transformed AUC₀₋ₜ of the oral dosage form to a geometric mean of logarithmic transformed AUC₀₋ₜ of COMPOUND I reference drug of about 0.80 to about 1.25.

In yet another embodiment, the oral dosage form comprises COMPOUND I, or a pharmaceutically acceptable salt thereof, comprising 0.3 to about 1% of the R isomer based on the total amount of S and R isomers or pharmaceutically acceptable salt thereof, wherein the oral dosage form exhibits a ratio of a geometric mean of logarithmic transformed Cₘₐₓ of the dosage form to a geometric mean of logarithmic transformed Cₘₐₓ of COMPOUND I reference drug of about 0.70 to about 1.43.

In yet another embodiment, the oral dosage form comprises COMPOUND I, or a pharmaceutically acceptable salt thereof, comprising 0.3 to about 1% of the R isomer based on the total amount of S and R isomers or pharmaceutically acceptable salt thereof, wherein the oral dosage form exhibits a ratio of a geometric mean of logarithmic transformed Cₘₐₓ of the oral dosage form to a geometric mean of logarithmic transformed Cₘₐₓ of COMPOUND I reference drug of about 0.80 to about 1.25.

COMPOUND I can be formulated into an oral dosage form that provides a Tₘₐₓ for healthy human subjects from about 15 minutes to about 3 hours, with the mean Tₘₐₓ value at about 1 hour under the fasting conditions, and about 2 hours under fed conditions. This dosage form provides an elimination half-life (t_{1/2}) at from about 3 to about 9 hours, with mean values at about 6 hours.

Also disclosed herein are COMPOUND I oral dosage forms that provide a quick therapeutic onset with average in less than about 30 minutes, such as a quick dissolving dosage forms or oral disintegrating tablets.

Also disclosed herein are COMPOUND I oral dosage forms that provide a prolonged active agent release, such as extended-release dosage forms with sleep maintenance for up to about 16 hours, specifically about 8 to about 12 hours.

In one embodiment, the dosage form exhibits an in vitro dissolution profile substantially corresponding to the profile exhibited by the reference drug according to New Drug Application NO. 021476 when tested in a similar fashion.

A dissolution profile is a plot of the cumulative amount of active agent released as a function of time. A dissolution profile can be measured utilizing the Drug Release Test <724>, which incorporates standard test USP 26 (Test <711>) which are both incorporated herein by reference, or by other test methods or conditions. A profile is characterized by the test conditions selected such as, for example, apparatus type (e.g. basket apparatus 1, paddle apparatus 2, reciprocating cylinder apparatus 3, flow through cell apparatus 4), shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A first dissolution profile can be measured at a pH level approximating that of the stomach. A second dissolution profile can be measured at a pH level approximating that of one point in the intestine or several pH levels approximating multiple points in the intestine.

A highly acidic pH may simulate the stomach and a less acidic to basic pH may simulate the intestine. By the term “highly acidic pH”: it is meant a pH of about 1 to about 4. By the term “less acidic to basic pH” is meant a pH of greater than about 4 to about 7.5, specifically about 6 to about 7.5. A pH of about 1.2 can be used to simulate the pH of the stomach. A pH of about 6 to about 7.5, specifically about 6.8, can be used to simulate the pH of the intestine.

In one embodiment, an oral dosage form comprises COMPOUND I, or a pharmaceutically acceptable salt thereof, comprising 0.3 to about 1% of the R isomer based on the total amount of S and R isomers or pharmaceutically acceptable salt thereof, wherein a dissolution profile of the composition is substantially the same as a dissolution profile of an equivalent strength of a reference drug according to New Drug Application No. 021476. The dissolution profile can be determined using the conditions according to USP 30 <711> test method 2 (paddle) or test method 1 basket, using of a certain volume of a dissolution medium at 37° C.±0.5° C., and specified paddle or shaft speed. The dissolution medium can be purified water, 0.1N HCl, 0.1N NaOH, 7.5 pH buffer, 6.8 pH buffer, 0.5% sodium dodecyl sulfate, and the like.

In another embodiment, an oral dosage form comprises COMPOUND I, or a pharmaceutically acceptable salt thereof, comprising 0.3 to about 1% of the R isomer based on the total amount of S and R isomers or pharmaceutically acceptable salt thereof wherein the composition exhibits a dissolution profile after combining the composition with 500 or 900 ml of purified water, 0.1N HCl, 0.1N NaOH, 7.5 pH buffer, 6.8 pH buffer, or 0.5% sodium dodecyl sulfate at 37° C.±0.5° C. according to USP 30 <711> Apparatus I at 100 rpm (or Apparatus II at 50 rpm), wherein about 70 to about 100 percent of the total amount of COMPOUND I is released after 1.5 hours; specifically about 70 to about 100 percent of the total amount of COMPOUND I is released after 1.0 hour; and yet more specifically about 70 to about 100 percent of the total amount of COMPOUND I is released after 45 minutes.

In one embodiment, an oral dosage form comprises COMPOUND I, or a pharmaceutically acceptable salt thereof, comprising 0.3 to about 1% of the R isomer based on the total amount of S and R isomers or pharmaceutically acceptable salt thereof, wherein a dissolution profile of the composition is substantially the same as a dissolution profile of an equivalent strength of a reference drug according to New Drug Application No. 021476. Specifically, the oral dosage form comprises COMPOUND I, or a pharmaceutically acceptable salt thereof, comprising 0.3 to about 1% of the R isomer based on the total amount of S and R isomers or pharmaceutically acceptable salt thereof wherein the composition exhibits a dissolution profile after combining the composition with 500 or 900 ml of a dissolution medium selected from (1) 0.1 N HCl or Simulated Gastric Fluid USP without enzymes; (2) a pH 4.5 buffer; or (3) a pH 6.8 buffer or Simulated Intestinal Fluid USP without enzymes; at 37° C.±0.5° C. according to USP 30 <711> Apparatus I at 100 rpm (or Apparatus II at 50 rpm), no less than 85 wt. % of the total amount of COMPOUND I is released after 30 minutes.

In another embodiment, a controlled-release oral dosage form comprises COMPOUND I, or a pharmaceutically acceptable salt thereof, comprising 0.3 to about 1% of the R isomer based on the total amount of S and R isomers or pharmaceutically acceptable salt thereof wherein the composition exhibits a dissolution profile after combining the composition with 500 or 900 ml of purified water, 0.1N HCl, 0.1N NaOH, 7.5 pH buffer, 6.8 pH buffer, or 0.5% sodium dodecyl sulfate at 37° C.±0.5° C. according to USP 30 <711> Apparatus I at 100 rpm (or Apparatus II at 50 rpm), wherein about 0.0001 to about 70 percent of the total amount of COMPOUND I is released after 1.0 hour and about 80 to about 100 percent of the total amount of COMPOUND I is released after 16.0 hours; and more specifically about 0.0001 to about 70 percent of the total amount of COMPOUND I is released after 2.0 hours and about 80 to about 100 percent of the total amount of COMPOUND I is released after 8.0 hours.

In one embodiment, the immediate release oral dosage form meets the criteria for a Biopharmaceutics Classification System wavier (“BCS Waiver”) according to the Guidance for Industry Waiver of In Vivo Bioavailability and Bioequivalence Studies for Immediate-Release Solid Oral Dosage Forms Based on a Biopharmaceutics Classification System, U.S. Department of Health and Human Services Food and Drug Administration Center for Drug Evaluation and Research (CDER) August 2000, which is incorporated herein in its entirety.

The waiver allows for sponsors of investigational new drug applications, new drug applications, abbreviated new drug applications, and supplements to these applications to request a waiver of in vivo bioavailability or bioequivalence studies for immediate release solid oral dosage forms based on the so-called Biopharmaceutics Classification System. The Biopharmaceutics Classification System classifies active agents based on their aqueous solubility and intestinal permeability. Further taken into consideration is the dissolution of the dosage form. Thus, the Biopharmaceutics Classification System involves dissolution of the dosage form, solubility of the active agent, and intestinal permeability of the active agent. Biowaivers may be allowed under the Biopharmaceutics Classification System for highly soluble and highly permeable active agents in immediate release solid oral dosage forms that exhibit rapid in vitro dissolution using the test methods outlined in 21 CFR 320.22(e).

Solubility of the active agent is tested using the highest dose strength of the immediate release product that is the subject of the biowaiver request. An active agent is considered highly soluble when the highest dose strength is soluble in 250 ml or less of aqueous media over the pH range of 1-7.5.

The permeability of the active agent is based indirectly on the extent of absorption (fraction of dose absorbed, not systemic bioavailability) of a drug substance in humans and directly on measurements of the rate of mass transfer across human intestinal membrane. Alternatively, nonhuman systems capable of predicting the extent of drug absorption in humans can be used (e.g., in vitro epithelial cell culture methods). In the absence of evidence suggesting instability in the gastrointestinal tract, a drug substance is considered to be highly permeable when the extent of absorption in humans is determined to be 90% or more of an administered dose based on a mass balance determination or in comparison to an intravenous reference dose.

An immediate release dosage form is considered rapidly dissolving when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

Under the Biopharmaceutics Classification System the equilibrium solubility of a drug substance under physiological pH conditions is to be determined. The pH-solubility profile of the test drug substance is determined at 37±1° C. in aqueous media with a pH in the range of 1-7.5. A sufficient number of pH conditions are evaluated to accurately define the pH-solubility profile. The number of pH conditions for a solubility determination can be based on the ionization characteristics of the test drug substance. For example, when the pKa of a drug is in the range of 3-5, solubility is determined at pH=pKa, pH=pKa+1, pH=pKa−1, and at pH=1 and 7.5. A minimum of three replicate determinations of solubility in each pH condition is recommended. Depending on study variability, additional replication may be necessary to provide a reliable estimate of solubility. Standard buffer solutions described in the USP are considered appropriate for use in solubility studies. If these buffers are not suitable for physical or chemical reasons, other buffer solutions can be used. Solution pH is verified after addition of the drug substance to a buffer. Methods other than the traditional shake-flask method, such as acid or base titration methods, can also be used with justification to support the ability of such methods to predict equilibrium solubility of the test drug substance. Concentration of the drug substance in selected buffers (or pH conditions) is determined using a validated stability-indicating assay that can distinguish the drug substance from its degradation products.

The solubility class is determined by calculating the volume of an aqueous medium sufficient to dissolve the highest dose strength in the pH range of 1-7.5. A drug substance is classified as highly soluble when the highest dose strength is soluble in ≦250 ml of aqueous media over the pH range of 1-7.5.

The permeability class of a drug substance can be determined in human subjects using mass balance, absolute bioavailability, or intestinal perfusion approaches. Recommended methods not involving human subjects include in vivo or in situ intestinal perfusion in a suitable animal model (e.g., rats), and/or in vitro permeability methods using excised intestinal tissues, or monolayers of suitable epithelial cells. In many cases, a single method may be sufficient (e.g., when the absolute bioavailability is 90% or more, or when 90% or more of the administered drug is recovered in urine). When a single method fails to conclusively demonstrate a permeability classification, two different methods may be advisable. Chemical structure and/or certain physicochemical attributes of a drug substance (e.g., partition coefficient in suitable systems) can provide useful information about its permeability characteristics.

One approach to pharmacokinetic studies in humans includes mass balance studies using unlabeled, stable isotopes or a radio labeled drug substance to document the extent of absorption of a drug. Depending on the variability of the studies, a sufficient number of subjects are enrolled to provide a reliable estimate of extent of absorption. Because this method can provide highly variable estimates of drug absorption for many drugs, other methods described below may be preferable. Another approach to pharmacokinetic studies in humans includes absolute bioavailability studies using intravenous administration as a reference. Depending on the variability of the studies, a sufficient number of subjects are enrolled in a study to provide a reliable estimate of the extent of absorption. When the absolute bioavailability of a drug is shown to be 90% or more, additional data to document drug stability in the gastrointestinal fluid is not necessary.

The following methods can be used to determine the permeability of a drug substance from the gastrointestinal tract: (1) in vivo intestinal perfusion studies in humans; (2) in vivo or in situ intestinal perfusion studies using suitable animal models; (3) in vitro permeation studies using excised human or animal intestinal tissues; or (4) in vitro permeation studies across a monolayer of cultured epithelial cells.

In vivo or in situ animal models and in vitro methods, such as those using cultured monolayers of animal or human epithelial cells, are considered appropriate for passively transported drugs. The observed low permeability of some drug substances in humans could be caused by efflux of drugs via membrane transporters such as P-glycoprotein (P-gp). When the efflux transporters are absent in these models, or their degree of expression is low compared to that in humans, there may be a greater likelihood of misclassification of permeability class for a drug subject to efflux compared to a drug transported passively. Expression of known transporters in selected study systems are characterized. Functional expression of efflux systems (e.g., P-gp) can be demonstrated with techniques such as bidirectional transport studies, demonstrating a higher rate of transport in the basolateral-to-apical direction as compared to apical-to-basolateral direction using selected model drugs or chemicals at concentrations that do not saturate the efflux system (e.g., cyclosporin A, vinblastine, rhodamine 123). It is recommended to limit the use of nonhuman permeability test methods for drug substances that are transported by passive mechanisms. Pharmacokinetic studies on dose linearity or proportionality may provide useful information for evaluating the relevance of observed in vitro efflux of a drug. For example, there may be fewer concerns associated with the use of in vitro methods for a drug that has a higher rate of transport in the basolateral-to-apical direction at low drug concentrations but exhibits linear pharmacokinetics in humans.

An apparent passive transport mechanism can be assumed when one of the following conditions is satisfied: i) linear (pharmacokinetic) relationship between the dose (e.g., relevant clinical dose range) and measures of BA (area under the concentration-time curve) of a drug is demonstrated in humans; ii) lack of dependence of the measured in vivo or in situ permeability is demonstrated in an animal model on initial drug concentration (e.g., 0.01, 0.1, and 1 times the highest dose strength dissolved in 250 ml) in the perfusion fluid; or iii) lack of dependence of the measured in vitro permeability on initial drug concentration (e.g., 0.01, 0.1, and 1 times the highest dose strength dissolved in 250 ml) is demonstrated in donor fluid and transport direction (e.g., no statistically significant difference in the rate of transport between the apical-to-basolateral and basolateral-to-apical direction for the drug concentrations selected) using a suitable in vitro cell culture method that has been shown to express known efflux transporters (e.g., P-gp).

To demonstrate suitability of a permeability method, a rank-order relationship between test permeability values and the extent of drug absorption data in human subjects are established using a sufficient number of model drugs. For in vivo intestinal perfusion studies in humans, six model drugs are recommended. For in vivo or in situ intestinal perfusion studies in animals and for in vitro cell culture methods, twenty model drugs are recommended. Depending on study variability, a sufficient number of subjects, animals, excised tissue samples, or cell monolayers are used in a study to provide a reliable estimate of drug permeability. This relationship allows differentiation between drug substances of low and high intestinal permeability attributes.

After demonstrating suitability of a method and maintaining the same study protocol, it is not necessary to retest all selected model drugs for subsequent studies intended to classify a drug substance. Instead, a low and a high permeability model drug can be used as internal standards (i.e., included in the perfusion fluid or donor fluid along with the test drug substance). These two internal standards are in addition to the fluid volume marker (or a zero permeability compound such as PEG 4000) that is included in certain types of perfusion techniques (e.g., closed loop techniques). The choice of internal standards is based on compatibility with the test drug substance (i.e., they do not exhibit any significant physical, chemical, or permeation interactions). When it is not feasible to follow this protocol, the permeability of internal standards can be determined in the same subjects, animals, tissues, or monolayers, following evaluation of the test drug substance. The permeability values of the two internal standards do not differ significantly between different tests, including those conducted to demonstrate suitability of the method. At the end of an in situ or in vitro test, the amount of drug in the membrane is determined.

For a given test method with set conditions, selection of a high permeability internal standard with permeability in close proximity to the low/high permeability class boundary may facilitate classification of a test drug substance. For instance, a test drug substance may be determined to be highly permeable when its permeability value is equal to or greater than that of the selected internal standard with high permeability.

Determining the extent of absorption in humans based on mass balance studies using total radioactivity in urine does not take into consideration the extent of degradation of a drug in the gastrointestinal fluid prior to intestinal membrane permeation. In addition, some methods for determining permeability could be based on loss or clearance of a drug from fluids perfused into the human and/or animal gastrointestinal tract either in vivo or in situ. Documenting the fact that drug loss from the gastrointestinal tract arises from intestinal membrane permeation, rather than a degradation process, will help establish permeability. Stability in the gastrointestinal tract may be documented using gastric and intestinal fluids obtained from human subjects. Drug solutions in these fluids are incubated at 37° C. for a period that is representative of in vivo drug contact with these fluids; for example, 1 hour in gastric fluid and 3 hours in intestinal fluid. Drug concentrations are then be determined using a validated stability-indicating assay method. Significant degradation (>5%) of a drug in this protocol could suggest potential instability. Obtaining gastrointestinal fluids from human subjects requires intubation and may be difficult in some cases. Use of gastrointestinal fluids from suitable animal models and/or simulated fluids such as Gastric and Intestinal Fluids USP can be substituted when properly justified.

Dissolution testing for the biowaiver is carried out in USP Apparatus I at 100 rpm or Apparatus II at 50 rpm using 900 ml of the following dissolution media: (1) 0.1 N HCl or Simulated Gastric Fluid USP without enzymes; (2) a pH 4.5 buffer; and (3) a pH 6.8 buffer or Simulated Intestinal Fluid USP without enzymes. For capsules and tablets with gelatin coating, Simulated Gastric and Intestinal Fluids USP (with enzymes) can be used.

Dissolution testing apparatus used in this evaluation conform to the requirements in USP (<711> Dissolution). Selection of the dissolution testing apparatus (USP Apparatus I or II) during drug development is based on a comparison of in vitro dissolution and in vivo pharmacokinetic data available for the product. The USP Apparatus I (basket method) is generally used for capsules and products that tend to float, and USP Apparatus II (paddle method) is generally used for tablets. For some tablet dosage forms, in vitro (but not in vivo) dissolution may be slow due to the manner in which the disintegrated product settles at the bottom of a dissolution vessel. In such situations, USP Apparatus I may be preferred over Apparatus II. If the testing conditions need to be modified to better reflect rapid in vivo dissolution (e.g., use of a different rotating speed), such modifications can be justified by comparing in vitro dissolution with in vivo absorption data (e.g., a relative bioavailability study using a simple aqueous solution as the reference product).

A minimum of twelve dosage units of a drug product are evaluated to support a biowaiver request. Samples are collected at a sufficient number of intervals to characterize the dissolution profile of the drug product (e.g., 10, 15, 20, and 30 minutes).

When comparing the test and reference products, dissolution profiles are compared using a similarity factor (f₂). The similarity factor is a logarithmic reciprocal square root transformation of the sum of squared error and is a measurement of the similarity in the percent (%) of dissolution between the two curves. f₂=50·log {[1+(1/n)Σₜ₌ₜⁿ(Rₜ−Tₜ)²]^{-0.5}·100} Two dissolution profiles are considered similar when the f₂ value is ≧50. To allow the use of mean data, the coefficient of variation is not more than 20% at the earlier time points (e.g., 10 minutes), and not more than 10% at other time points. Note that when both test and reference products dissolve 85% or more of the label amount of the drug in ≦15 minutes using all three dissolution media recommended above, the profile comparison with an f₂ test is unnecessary.

For new drug applications, evidence demonstrating in vivo bioavailability is included in the application (21 CFR 320.21(a)). A specific objective is to establish in vivo performance of the dosage form used in the clinical studies that provided primary evidence of efficacy and safety. The relative bioavailability of an immediate release solid oral dosage form can be determined by comparison with an oral solution, suspension, or intravenous injection (21 CFR 320.25 (d)(2) and 320.25 (d)(3)). The bioavailability of the clinical trial dosage form can be optimized during the investigational new drug period.

Once the in vivo bioavailability of a formulation is established during the investigational new drug period, waivers of subsequent in vivo bioequivalent studies, following major changes in components, composition, and/or method of manufacture may be possible using the Biopharmaceutics Classification System. Biopharmaceutics Classification System-based biowaivers are applicable to the to-be-marketed formulation when changes in components, composition, and/or method of manufacture occur to the clinical trial formulation, as long as the dosage forms have rapid and similar in vitro dissolution profiles. This approach is useful when the drug substance is highly soluble and highly permeable, and the formulations pre- and postchange are pharmaceutical equivalents (under the definition at 21 CFR 320.1 (c)).

Biopharmaceutics Classification System-based biowaivers can be requested for rapidly dissolving immediate release test products containing highly soluble and highly permeable drug substances, provided that the reference listed drug product is also rapidly dissolving and the test product exhibits similar dissolution profiles to the reference listed drug product. This approach is useful when the test and reference dosage forms are pharmaceutical equivalents. The choice of dissolution apparatus (USP Apparatus I or II) is the same as that established for the reference listed drug product.

The oral dosage forms disclosed herein can be used to treat sleep disorders, for example, insomnia, disturbed sleep patterns, or providing sleep induction before surgical procedures or in disturbed or anxious states. The term “treating sleep disorders” means relief from insomnia, disturbed sleep patterns, or providing sleep induction before surgical procedures or in disturbed or anxious states. The oral dosage form can also be used to relieve the symptoms of epilepsy, which include, but are not limited to, altered consciousness, altered motor activity, autonomic responses, inappropriate behavior patterns, seizures including tonic or clonic jerking of extremities, emotional stress, sense of terror, uneasiness, nervousness, headache, fatigue, auditory hallucinations, aggressive outbursts, acute skeletal muscle spasm, and spasticity. The term “treating convulsive disorders” means relief from the symptoms of epilepsy, which include, but are not limited to, altered consciousness, altered motor activity, autonomic responses, inappropriate behavior patterns, seizures including tonic or clonic jerking of extremities, emotional stress, sense of terror, uneasiness, nervousness, headache, fatigue, auditory hallucinations, aggressive outbursts, acute skeletal muscle spasm, and spasticity.

In one embodiment, the oral dosage form is used to relieve the symptoms of insomnia, for example, the oral dosage form increases sleep time and improves sleep quality, and decreases the number of episodes of waking at night and of early morning awakening.

In one embodiment, a method of treating a patient in need of COMPOUND I therapy, comprises administering an oral dosage form comprising a therapeutically effective amount of COMPOUND I, or a pharmaceutically acceptable salt thereof, comprising 0.3 to about 1% of the R isomer based on the total amount of S and R isomers or pharmaceutically acceptable salt thereof; and a pharmaceutically acceptable excipient. The method of treating includes treating insomnia.

In another embodiment, a method of treating a patient in need of COMPOUND I therapy, comprises administering an oral dosage form comprising a therapeutically effective amount of COMPOUND I, or a pharmaceutically acceptable salt thereof, comprising 0.3 to about 1% of the R isomer based on the total amount of S and R isomers or pharmaceutically acceptable salt thereof; and a pharmaceutically acceptable excipient; wherein the amount of R isomer present in the COMPOUND I remains substantially unchanged between an initial time point and after storage of the COMPOUND I at about 25° C. and about 60% relative humidity for 12 months. The method of treating includes treating insomnia.

In yet another embodiment, a method of treating a patient in need of COMPOUND I therapy, comprises administering an oral dosage form comprising a therapeutically effective amount of COMPOUND I, or a pharmaceutically acceptable salt thereof, comprising 0.3 to about 1% of the R isomer based on the total amount of S and R isomers or pharmaceutically acceptable salt thereof; and a pharmaceutically acceptable excipient; wherein the COMPOUND I has an average particle size about 0.1 to about 500 micrometers.

In one embodiment, a method for detecting the presence of R isomer present in a sample of Compound I or a pharmaceutically acceptable salt thereof, comprises performing high performance liquid chromatography on the sample using a chiral column.

In another embodiment, method for detecting the presence of R isomer present in a sample of Compound I or a pharmaceutically acceptable salt thereof, comprises performing capillary electrophoresis on the sample.

### EXAMPLES

### Example 1

### High Performance Liquid Chromatography (HPLC) Analysis of COMPOUND I

Samples of COMPOUND I are analyzed for quantities of the S and R isomer according to the following parameters:

TABLE 1

| | |
|---|---|
| Equipment | Waters Alliance HPLC |
| Detector | 300 nanometer |
| Column | 250 × 4.6 millimeter, Chiralcel, Cellulose tris (3,5- |
| | dimethylphenylcarbamate) on 10 micrometer silica gel |
| | substance (Manufacturer Chiral Technologies Inc.) |
| Mobile phase | Ethanol, 200 proof (Sigma-Aldrich) |
| Flow rate | 0.5 milliliter |
| Pressure | about 280 pounds per square inch |
| Injection volume | 10 microliter |
| Standard/sample | 0.5 milligram/milliliter in ethanol |
| concentration | |

Several batches of COMPOUND I are prepared and tested for quantities of the S and R isomer and provided in Table 2.

TABLE 2

| Sample | S isomer (Area %) | R isomer (Area %) |
|---|---|---|
| A | 99.55 | 0.28 |
| B | 99.55 | 0.35 |
| C | 99.91 | 0.010 |
| D | 99.91 | 0.020 |
| E | 99.90 | 0.020 |

### Example 2

### Capillary Electrophoresis Analysis of COMPOUND I

Samples of COMPOUND I are analyzed for quantities of the S and R isomer according to the following parameters:

TABLE 3

| | |
|---|---|
| Equipment | Capillary Electrophoresis System |
| Capillary | Extended Light Path, internal diameter 50 μm, |
| | effective length 40 cm |
| Electrolyte | 0.1 M H₃PO₄ adjust with triethanolamine to |
| | pH = 2.74 |
| Chiral Selector | 7.23 mg of β-cyclodextrin sulphated sodium |
| | salt in 1.0 mL of the filtered electrolyte. |
| Solvent | Acetonitrile:Water (1:4) |
| Standard Solution | 5.0 mg of Compound I and 0.1 mg of Compound II |
| | in 5 mL of Solvent |
| Sample Solution | 5.0 mg of sample in 5.0 mL of Solvent |
| Lift Offset | 4 mm |
| Capillary | 25° C. |
| Temperature | |
| Pre-conditioning | 2.0 min flush with 1 M NaOH |
| | 0.5 minute wait |
| | 2.0 min flush with Chiral Selector |
| Injection | 200 mbar · s (50 mbar for 4 seconds) |
| Polarity | Positive |
| CE Time Table | 0.2 minutes, 30 kV |
| Run Time | 12 minutes |
| Post-conditioning | 2.0 minute flush with Electrolyte |
| Detector | Ultrviolet Spectrophotometry at 301 nm |

### Example 3

### COMPOUND I Tablets, 1 mg, 2 mg, 3 mg

COMPOUND I tablets are prepared using the COMPOUND I of sample B from Example 1 above. The general formulation is provided in Table 4 below.

TABLE 4

| | | Amount per tablet (milligrams) | |
|---|---|---|---|
| | | wt % based on the total weight of | |
| | Component | the tablet without film coating. | |
| 1 | COMPOUND I (Sample B, | 1-3 | milligrams |
| | Example 1) | | |
| 2 | Tablet Diluent | 1-90 | wt % |
| 3 | Disintegrant | 1-20 | wt % |
| 4 | Glidant | 0-5 | wt % |
| 5 | Lubricant | 1-5 | wt % |

The components 1-3 are mixed followed by the addition of a lubricant and optional glidant to form a final mixture. The final mixture is compressed into tablets using standard compression equipment known in the art to result in tablets containing 1-3 milligrams of COMPOUND I each. The tablets can optionally be film coated with appropriate film coatings (e.g., film former (hydroxypropyl methylcellulose), plasticizer (polyethylene glycol, Triacetin), and optional colorant) such as those sold under the Opadry mark available from Colorcon.

### Example 4

### Orally Disintegrating Tablet

COMPOUND I orally disintegrating tablets are prepared using the COMPOUND I of sample B from Example 1 above. The general formulations are provided in Table 5 below.

TABLE 5

| | | Amount per tablet (weight percent) | | |
|---|---|---|---|---|
| | Component | A | B | C |
| 1 | COMPOUND I | 1-3 milligrams | 1-3 milligrams | 1-3 milligrams |
| | (Sample B, | per tablet | per tablet | per tablet |
| | Example 1) | | | |
| 2 | Mannitol | 1-90% | 1-90% | — |
| 3 | Microcrystalline | 1-90% | 1-90% | 1-90% |
| | cellulose | | | |
| 4 | Pregelatinized | 1-30% | — | — |
| | starch | | | |
| 5 | Sodium starch | 1-30% | — | — |
| | glycolate | | | |
| 6 | Croscarmellose | — | — | 1-30% |
| | sodium | | | |
| 7 | Corn starch | — | 1-30% | — |
| 8 | Magnesium | 0.001-10 | 0.001-10 | 0.001-10 |
| | stearate | | | |
| 9 | Methacrylic acid | — | 1-10% | — |
| | Copolymer | | | |
| 10 | Butylated | 1-10% | — | — |
| | methacrylate | | | |
| | Copolymer | | | |
| 11 | Crospovidone | 1-90% | 1-90% | 1-90% |
| 12 | Sweetener | 0.5-30% | 0.5-30% | 0.5-30% |
| | (xylitol, | | | |
| | sucralose, | | | |
| | sucrose, | | | |
| | acesulfame | | | |
| | potassium, or | | | |
| | aspartame) | | | |
| 13 | Tartaric acid | — | — | 1-20% |
| 14 | Citric acid | 1-25% | — | — |
| 15 | Sodium | 1-25% | — | — |
| | bicarbonate | | | |
| 16 | Calcium silicate | — | — | 0-3% |
| 17 | Colloidal | 0-3% | 0-3% | 0-3% |
| | silicon | | | |
| | dioxide | | | |
| 18 | Flavorant | 0-3% | 0-3% | 0-3% |
| 19 | Colorant | 0-3% | 0-3% | 0-3% |
| | Total | 100% | 100% | 100% |

Formulations A and B are orally disintegrating tablets containing coated particles of COMPOUND I in a quick disintegrating tablet matrix. Formulation A contains a water or saliva activated effervescent disintegrant of citric acid and sodium bicarbonate. Particles of COMPOUND I are prepared by granulating COMPOUND I and microcrystalline cellulose and crospovidone. The particles are coated with butylated methacrylate copolymer as a protective coating. The resulting coated particles are combined with mannitol, pregelatinized starch, sodium starch glycolate, aspartame, citric acid, sodium bicarbonate, colloidal silicon dioxide, flavorant, colorant, and magnesium stearate and formed into tablets.

Formulation B also contains coated particles of COMPOUND I prepared by granulating COMPOUND I and microcrystalline cellulose and crospovidone to form particles. Methacrylic acid copolymer is used as the protective coating that is coated on the particles. The resulting coated particles are combined with mannitol, corn starch, sweetener, colloidal silicon dioxide, flavorant, colorant, and magnesium stearate and formed into tablets.

Formulation C is prepared by blending the components of Table 6 and forming into orally disintegrating tablets using techniques known to one of ordinary skill in the art.

### Example 5

### Disintegration Testing

The tablets of Example 4 are analyzed according to USP disintegration test <701> in purified water at a temperature of 37±2° C. The tablets disintegrate in less than three minutes.

The use of the terms “a” and “an” and “the” and similar referents (especially in the context of the following claims) are to be construed to cover both the singular and the plural, unless otherwise indicated herein or clearly contradicted by context. The term “or” means “and/or”. The endpoints of all ranges directed to the same component or property are inclusive and independently combinable. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein, the terms wt %, weight percent, percent by weight, etc. are equivalent and interchangeable.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. COMPOUND I, 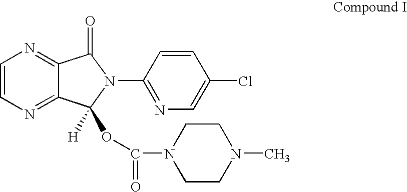
or a pharmaceutically acceptable salt thereof, comprising 0.3 to about 1% of the R isomer based on the total amount of S and R isomers or pharmaceutically acceptable salt thereof.

2. (canceled)

3. The COMPOUND I of claim 1,
wherein the COMPOUND I has an average particle size about 0.1 to about 500 micrometers.

4.-5. (canceled)

6. The COMPOUND I of claim 3 having an average particle size about 25 to about 100 micrometers.

7.-10. (canceled)

11. The COMPOUND I of claim 1, wherein the amount of R isomer varies by less than about 0.5% between
an initial time point and after storage of the COMPOUND I at about 25° C. and about 60% relative humidity for 12 months;
an initial time point and after storage of the COMPOUND I at about 40° C. and about 75% relative humidity for 30 days;
an initial time point and after storage of the COMPOUND I at about 40° C. and about 75% relative humidity for 60 days;
an initial time point and after storage of the COMPOUND I at about 40° C. and about 75% relative humidity for 90 days; or
an initial time point and after storage of the COMPOUND I at about 40° C. and about 75% relative humidity for 180 days or greater.

12. The COMPOUND I of claim 1, wherein the amount of R isomer varies by less than about 0.05% between
an initial time point and after storage of the COMPOUND I at about 25° C. and about 60% relative humidity for 12 months;
an initial time point and after storage of the COMPOUND I at about 40° C. and about 75% relative humidity for 30 days;
an initial time point and after storage of the COMPOUND I at about 40° C. and about 75% relative humidity for 60 days;
an initial time point and after storage of the COMPOUND I at about 40° C. and about 75% relative humidity for 90 days; or
an initial time point and after storage of the COMPOUND I at about 40° C. and about 75% relative humidity for 180 days or greater.

13. (canceled)

14. An oral dosage form, comprising:
a therapeutically effective amount of COMPOUND I, or a pharmaceutically acceptable salt thereof, comprising 0.3 to about 1% of the R isomer based on the total amount of S and R isomers or pharmaceutically acceptable salt thereof; and
a pharmaceutically acceptable excipient.

15. (canceled)

16. The oral dosage form of claim 14
wherein the COMPOUND I has an average particle size about 0.1 to about 500 micrometers.

17.-18. (canceled)

19. The oral dosage form of claim 16, wherein the COMPOUND I has an average particle size about 25 to about 100 micrometers.

20.-23. (canceled)

24. The oral dosage form of claim 14, wherein the amount of R isomer varies by less than about 0.5% between
an initial time point and after storage of the COMPOUND I at about 25° C. and about 60% relative humidity for 12 months;
an initial time point and after storage of the COMPOUND I at about 40° C. and about 75% relative humidity for 30 days;
an initial time point and after storage of the COMPOUND I at about 40° C. and about 75% relative humidity for 60 days;
an initial time point and after storage of the COMPOUND I at about 40° C. and about 75% relative humidity for 90 days; or
an initial time point and after storage of the COMPOUND I at about 40° C. and about 75% relative humidity for 180 days or greater.

25. The oral dosage form of claim 14, wherein the amount of R isomer varies by less than about 0.05% between
an initial time point and after storage of the COMPOUND I at about 25° C. and about 60% relative humidity for 12 months;
an initial time point and after storage of the COMPOUND I at about 40° C. and about 75% relative humidity for 30 days;
an initial time point and after storage of the COMPOUND I at about 40° C. and about 75% relative humidity for 60 days;
an initial time point and after storage of the COMPOUND I at about 40° C. and about 75% relative humidity for 90 days; or
an initial time point and after storage of the COMPOUND I at about 40° C. and about 75% relative humidity for 180 days or greater.

26. The oral dosage form of claim 14,
wherein the oral dosage form is bioequivalent to a reference listed drug according to New Drug Application No. 021476.

27.-35. (canceled)

36. An oral dosage form, comprising:
a therapeutically effective amount of COMPOUND I, or a pharmaceutically acceptable salt thereof, comprising 0.3 to about 1% of the R isomer based on the total amount of S and R isomers or pharmaceutically acceptable salt thereof; and
a pharmaceutically acceptable excipient;
wherein the amount of R isomer present in the COMPOUND I remains substantially unchanged between an initial time point and after storage of the COMPOUND I at about 25° C. and about 60% relative humidity for 12 months, and
wherein the COMPOUND I has an average particle size about 0.1 to about 500 micrometers.

37. (canceled)

38. The oral dosage form of claim 14, wherein the COMPOUND I is in the form of crystals, co-crystals, granules, microgranules, powders, pellets, amorphous solids, amorphous dispersions, or precipitates.

39. (canceled)

40. The oral dosage form of claim 1, wherein the oral dosage form is bioequivalent to a reference listed drug according to New Drug Application No. 021476.

41.-47. (canceled)

48. The oral dosage form of claim 14, wherein the oral dosage form comprises an immediate release oral dosage form meeting the criteria for a Biopharmaceutics Classification System wavier according to the Guidance for Industry Waiver of In Vivo Bioavailability and Bioequivalence Studies for Immediate-Release Solid Oral Dosage Forms Based on a Biopharmaceutics Classification System, U.S. Department of Health and Human Services Food and Drug Administration Center for Drug Evaluation and Research (CDER) August 2000.

49.-51. (canceled)

52. The oral dosage form of claim 14, wherein the oral dosage form exhibits a dissolution profile of the composition is substantially the same as a dissolution profile of an equivalent strength of a reference drug according to New Drug Application No. 021476.

53.-58. (canceled)

59. The oral dosage form of claim 14, formulated into a quick dissolving tablet, an orally disintegrating tablet, a chewable tablet, a monolithic tablet, a layered tablet, or a capsule.

60. A method of treating insomnia in a patient, comprising: administering the oral dosage form according to claim 14.

61. The COMPOUND I of claim 1, wherein the COMPOUND I exhibits an assay value of about 98% to about 102% as determined by high performance liquid chromatography, capillary electrophoresis, thin layer chromatography, or titration.

62. The oral dosage form of claim 14, wherein the COMPOUND I exhibits an assay value of about 98% to about 102% as determined by high performance liquid chromatography, capillary electrophoresis, thin layer chromatography, or titration.
